(19) United States
(12) Patent Application Publication
(10) Pub. No.: US 2007/0196462 A1
(43) Pub. Date: Aug. 23, 2007

(54) Composition and method for neuromuscular blockade

(75) Inventor: Evan C. Unger, Tucson, AZ (US)

Correspondence Address: DLA PIPER US LLP, 4365 EXECUTIVE DRIVE, SUITE 1100, SAN DIEGO, CA, 92121-2133, US

(73) Assignee: IMARX THERAPEUTICS, INC., Tucson, AZ (US)

(21) Appl. No.: 11/657,166

(22) Filed: Jan. 23, 2007

Related U.S. Application Data
(60) Provisional application No. 60/761,591, filed on Jan. 24, 2006.

(51) Int. Cl. A61K 39/08; A61K 9/127

(52) U.S. Cl. 424/450; 424/239.1; 977/907

(57) ABSTRACT

A neuromuscular blocking preparation for blocking and/or alleviating a vasospasm is provided, the preparation comprising a plurality of gas-or a gas precursor-filled microspheres, and a neuromuscular blocking agent. Methods for using such a preparation for blocking and/or alleviating a vasospasm are also provided.

### CROSS-REFERENCE TO RELATED APPLICATIONS

This application claims priority under 35 U.S.C. §119(e) to U.S. Patent Application Ser. No. 60/761,591 filed Jan. 24, 2006, which is herein incorporated by reference in its entirety.

### BACKGROUND

1. Field of the Invention

The present invention relates generally to the field of medical compositions and methods for treating vasospasms. More specifically, gas-or gas precursor-filled microspheres are provided having incorporated therein, onto the surface thereof, and/or co-administered with, a neuromuscular blocking agent to block and/or reduce the incidence of vasospasms in vivo by causing vascular neuromuscular blockade.

2. Background Information

The human cardiovascular system is a closed tubular system in which blood, propelled by a muscular heart, flows through vessels to and from all parts of the body. Two circuits, the pulmonary and the systemic, consist of arterial, capillary, and venous components.

Multiple problems and/or events may arise within the human cardiovascular system. For example, various bleeds may ensue, ruptures of various vessels may occur, etc. These events may lead to the occurrence of vasospasm(s), i.e., a constriction or narrowing of blood vessels. Commonly, vasospasms occur after a hemorrhage. In this case, the vessel constriction is prompted by chemical signals from the escaped blood as it breaks down.

Symptomatic vasospasm, also referred to as delayed ischemic deficits, delayed ischemic neurological defects, and cerebrovascular spasm, is the most common serious complication after aneurysmal rupture and is the leading cause of death for patients who survive the rupture. In fact, vasospasm-induced narrowing of cerebral vessels is estimated to occur in 70% to 90% of patients hospitalized for subarachnoid hemorrhage (SAH). Vasospasm is symptomatic in 30% of these patients.

Vasospasm has been described as a sustained arterial contraction unresponsive to vasodilator drugs. This condition is commonly classified as either angiographic or clinical. Angiographic vasospasm refers to visible narrowing of the dye column in an artery, as shown on cerebral angiograms. Clinical vasospasm is the functional manifestation of cerebral ischemia produced by this arterial narrowing.

Episodes of vasospasm can be benign or devastating. Among patients with SAH, 70% have angiographic evidence of vasospasm but no clinical evidence of vasospasm. The remainder (those with clinical vasospasm) have changes in findings on neurological examination. Clinically, the onset of new or worsening neurological signs or symptoms is the most reliable indicator of vasospasm. Assessment findings may be subtle and include headache, lethargy, and intermittent disorientation, which can progress to focal neurological deficits such as hemiparesis and speech dysfunction. Deficits vary according to the degree of vessel constriction and the cerebral artery affected. If the reduction in cerebral blood flow is severe or remains untreated, permanent disability and even death can occur.

Therefore, there is a need for an improved treatment for vasospasms, so as to reduce, minimize and/or alleviated the above-noted deficits.

### BRIEF DESCRIPTION OF THE DRAWINGS

FIG. 1 is a schematic illustration of a neuromuscular blocking preparation according to one embodiment of the present invention.

### SUMMARY

According to one embodiment of the present invention, a neuromuscular blocking preparation for blocking and/or alleviating a vasospasm is provided, the preparation comprising a plurality of gas-or a gas precursor-filled microspheres, and a neuromuscular blocking agent.

According to other embodiments of the present invention, various methods of treating vasospasm in a mammal are provided, comprising administering a combination of a plurality of gas-or a gas precursor-filled microspheres and a neuromuscular blocking agent to a patient in need of treatment, thereby blocking or alleviating a vasospasm.

### DETAILED DESCRIPTION

### 1. Terms and Definitions

The term “microsphere” is defined to refer to spherical or sphere-like particles having diameter between about 1 nanometer and 10 micrometers.

The term “gas-filled microsphere” is defined to refer to any microsphere including a gas or a gas precursor disposed therein in the amount equal to, or greater than, about 1 percent by volume of the microsphere.

The term “gas precursor” is defined to refer to a compound capable of changing phases from a liquid to a gas, typically, at a selected activation or transition temperature. Activation or transition temperature, and like terms, refer to the boiling point of the gaseous precursor, the temperature at which the liquid to gaseous phase transition of the gaseous precursor takes place.

The term “lipid” is defined to refer to a water-insoluble substance containing long chains of fatty acids.

The term “phospholipid” is defined to refer to a lipid that contains a group comprising an atom of phosphorous, for example, a phosphate group.

The term “at least co-administered” means that the neuromuscular blocking agent(s) may be applied to the surface of the gas-or gas precursor-filled microspheres or incorporated within the microspheres, or both applied to the surface of the microspheres and incorporated within the microspheres.

Additionally, the term “co-administered” as used herein includes administering the neuromuscular blocking agent immediately followed by gas-or gas precursor-filled microspheres or vice versa. The term “co-administered” also includes administering these components together (i.e., at the same time, with the same or different routes of administration) or essentially together (administering one component immediately followed by the administration of the other component). Any combination and/or derivation of the above is also included within the definition. For example, the neuromuscular blocking agent and the gas-or gas precursor-filled microspheres can be administered in a rapid succession, consecutively, in any order, such as when the time gap between the administration of each component is less than 10 seconds, e.g., less than 5 seconds, for example, 1 second. Compete simultaneity in administration, e.g., administering using two separate syringes at exactly the same moment in time is also contemplated.

### 2. Embodiments of the Invention

The present invention relates to a composition and a method for treating vasospasms by causing vascular neuromuscular blockade. This treatment of vasospasm offers an alternative to conventional treatments, which include, among other treatments, administering to a patient medications that increase blood pressure so to effectively force blood flow through the vasospasm so there is not an ischemic event at the site of the blood-deprived tissue located downstream of the vasospasm. Vasospasms often lead to downstream ischemic events, for example, cerebral ischemia in the brain. The present invention improves blood flow from the vasospasm state so as to reduce, minimize and/or eliminate ischemic events and consequences.

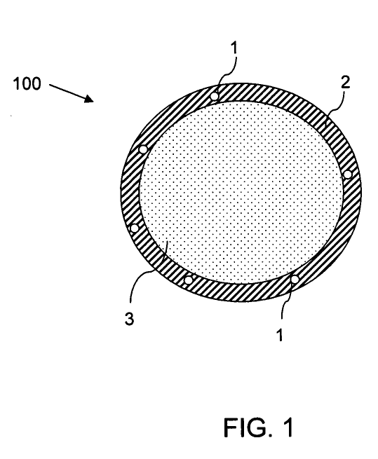

More specifically, and with the reference to FIG. 1, gas-or gas precursor-filled microspheres 100 are provided having incorporated therein, onto the surface thereof, and/or co-administered with, a neuromuscular blocking agent 1, to block and/or reduce the incidence of vasospasms in vivo. These microspheres 100 may range in size from about 1 nanometer to about 10 micrometers in diameter, for example, between about 100 nanometers to about 2 micrometers.

Typically, the gas-filled microsphere includes a shell 2 encompassing a gas or a gas precursor 3, or a combination of a gas and a gas precursor, the shell having an outer surface comprised of any material, such as a lipid, for example a phospholipid. The shell 2 can comprise liposomes, lipid coatings, emulsions and polymers. The liposomes may be formed as monolayers or bilayers and may or may not have a coating.

Accordingly, embodiments of the present invention utilize gas-or gas precursor-filled microspheres 100, including those discussed in the U.S. Pat. Nos. 6,071,495, 6,033,646, 5,770,222, 5,769,080, and 5,705,187. Each of these patents is incorporated herein by reference in their entirety, and any gas-or gas precursor-filled microspheres 100, or any combination thereof, can be used in the present invention. Some non-limiting examples of gas-or gas precursor-filled microspheres 100 that can be used are provided below.

More specifically, examples of lipids if used to create microspheres 100 include, but are not limited to, fatty acids, such as saturated or unsaturated organic acids that include, but are not limited to, molecules that have between 12 carbon atoms and 22 carbon atoms in either linear or branched form (e.g., lauric, myristic, palmitic acid, stearic acid, arachidonic acid, lauroleic, physeteric, myristoleic, palmitoleic, petroselinic, oleic, isolauric, isomyristic, isopalmitic, isostearic acids or isoprenoids), including lipids with ether and ester-linked fatty acids, lysolipids, phosphatidylcholine with both saturated and unsaturated lipids (e.g., dioleoylphosphatidylcholine, dimyristoylphosphatidylcholine, dipentadecanoylphosphatidylcholine, dilauroylphosphatidylcholine, dioleoylphosphatidylcholine, dipalmitoylphosphatidylcholine (DPPC), or distearoylphosphatidylcholine), phosphatidylethanolamines (e.g., dioleoylphosphatidylethanolamine), phosphatidylserine, phosphatidylglycerol, phosphatidylinositol, N-[1-(2,3-dioleoyloxy)propyl]-N,N,N-trimethylammonium chloride(DOTMA), 1,2-dioleoyloxy-3-(trimethylammonio)propane (DOTAP), 1,2-dioleoyl-3-(4′-trimethyl-ammonio)butanoyl-sn-glycerol (DOTB), sphingolipids (e.g., sphingomyelin), glycolipids (e.g., ganglioside GM1 and GM2), glucolipids, sulfatides, glycosphingolipids, phosphatidic acid, lipids bearing polymers such as polyethyleneglycol, chitin, hyaluronic acid or polyvinylpyrrolidone, lipids bearing saccharides (e.g., sulfonated mono-, di-, oligo- or polysaccharides), lipids bearing cholesterol or derivatives thereof (e.g., cholesterol sulfate or cholesterol hemisuccinate), tocopherol hemisuccinate, polymerized lipids, diacetyl phosphate, stearylamine, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3-β-yloxy)-1-thio-β-D-galactopyranoside, digalactosyldiglyceride, 6-(5-cholesten-3-β-yloxy)hexyl-6-amino-6-deoxy-1-thio-β-D-galacto pyranoside, 6-(5-cholesten-3-β-yloxy)hexyl-6-amino-6-deoxyl-1-thio-α-D-manno pyranoside, 12-(((7′-diethylaminocoumarin-3-yl)carbonyl)methylamino)-octadecanoic acid; N-[12-(((7′-diethylaminocoumarin-3-yl)carbonyl)methyl-amino)octadecanoyl]-2-aminopalmitic acid; cholesteryl)4′-trimethylammonio)butanoate, N-succinyldioleoylphosphatidylethanolamine, 1,2-dioleoyl-sn-glycerol, 1,2-dipalmitoyl-sn-3-succinylglycerol, 1,3-dipalmitoyl-2-succinylglycerol, 1-hexadecyl-2-palmitoylglycerophosphoethanolamine, palmitoylhomocysteine, and/or combinations thereof.

Lipids bearing hydrophilic polymers such as polyethyleneglycol (PEG), including and not limited to PEG 2,000 MW, 5,000 MW, and PEG 8,000 MW, are also useful for improving the stability and size distribution of the gaseous precursor-containing liposomes. PEGylated lipids, such as dipalmitoylphosphatidylethanolamine (DPPE) bearing PEG 5,000 MW, having various different mole ratios between PEG and the lipid, are also useful; for example a PEG/DPPE lipid having about 8 mole % DPPE can be used. One example of a lipid useful for entrapping gaseous precursors contains about 83 mole % DPPC, about 8 mole % DPPE-PEG 5,000 MW, and about 5 mole % dipalmitoylphosphatidic acid.

In addition, examples of compounds that can be used to create microspheres 100 include, but not limited to lauryltrimethylammonium bromide dodecyletyltrimethylammonium bromide, hexadecylmyristyltrimethylammonium bromide, tetradecylalkyldimethylbenzylammonium chloride (e.g., when the alkyl is C₁₂, C₁₄, or C₁₆), benzyldimethyldodecylammonium bromide or chloride, benzyldimethylhexadecylammonium bromide or chloride, benzyldimethyltetradecylammonium bromide or chloride, cetyldimethylethylammonium bromide or chloride, or cetylpyridinium bromide or chloride, or combinations thereof. Perfluorocarbons can be also used, such as pentafluoro octadecyl iodide, perfluorooctylbromide, perfluorodecalin, perfluorododecalin, perfluorooctyliodide, perfluorotripropylamine, or perfluorotributylamine, or combinations thereof. The perfluorocarbons may be entrapped in liposomes or stabilized in emulsions as is known to those having ordinary skill in the art.

If desired, either anionic or cationic lipids may be used if desired. In general, the molar ratio of a cationic lipid, if used, to a non-cationic lipid in the liposome may be, between about 2:1 and about 1:10, such as between about 1:1 and about 1:2.5, for example, about 1:1. In lieu of cationic lipids, lipids bearing cationic polymers such as polylysine, polyarginine (or their forms such as polyhomolysine or polyhomoarginine, respectively) or amphiphilic perfluoroalkylated bipyridines may also be used to construct the microspheres. Additionally, negatively charged lipids may be used, such as about 5 to 10 mole % phosphatidic acid. Other useful lipids or combinations thereof apparent to those skilled in the art which are in keeping with the spirit of the present invention are also encompassed by the present invention. For example, carbohydrate-bearing lipids may be employed, as described in U.S. Pat. No. 4,310,505, the disclosures of which is hereby incorporated herein by reference in its entirety.

Bioactive materials, such as peptides or proteins, may be incorporated into the lipid layer provided the peptides have sufficient lipophilicity or may be derivatized with alkyl or sterol groups for attachment to the lipid layer. Negatively charged peptides may be attached, for example, using cationic lipids or polymers described above.

One or more emulsifying or stabilizing agents may be included with the gaseous precursors to formulate the temperature activated gaseous precursor-filled microspheres 100. These agents help to maintain the size of the gaseous precursor-filled microsphere 100, and are also useful coating or stabilizing the microsphere 100, which results from the precursor. The optional stabilization of contrast agent-containing microspheres 100 is desirable to maximize the in vivo contrast effect.

Examples of gases or gas precursors 3 that can be used to create microspheres 100 include, but are not limited to, fluorine, perfluorocarbons (perfluoromethane, perfluoroethane, perfluoropropane, perfluorobutane, perfluoropentane, perfluorohexane, perfluorocyclobutane), sulfur hexafluoride, hexafluoropropylene, bromochlorofluoromethane, octafluoropropane, 1,1-dichlorofluoroethane, hexafluoroethane, hexafluoro-2-butyne, perfluoropentane, octafluoro-2-butene, hexafluorobuta-1,3-diene, octafluorocyclopentene, hexafluoroacetone, tetrafluoro allene, boron trifluoride, 1,2,3-trichloro-2-fluoro-1,3-butadiene, hexafluoro-1,3-butadiene, 1-fluorobutane, decafluorobutane, perfluoro-1-butene, perfluoro-2-butene, 2-chloro-1,1,1,4,4,4-hexafluoro-butyne, perfluoro-2-butyne, octafluorocyclobutane, perfluorocyclobutene, 1,1,1-trifluorodiazoethane, hexafluorodimethyl amine, perfluorodimethylamine, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1-chloro-1,1,2,2-tetrafluoro-ethane, 2-chloro, 1,1-difluoroethane, chloropentafluoroethane, dichlorotrifluoroethane, fluoroethane, hexafluoroethane, nitropentafluoroethane, nitrosopentafluoroethane, perfluoroethylamine, 1,1-dichloro-1,2-difluoroethylene, 1,2-difluoroethylene, methane-sulfonyl chloride-trifluoro, methane-sulfonyl fluoride-trifluoro, methane-(pentafluorothio)trifluoro, methane-bromo difluoro nitroso, methane-bromo fluoro, methane-bromo chloro-fluoro, methane-bromo-trifluoro, methane-chloro difluoro nitro, methane-chloro fluoro, methane-chloro trifluoro, methane-chloro-difluoro, methane-dibromo difluoro, methane-dichloro difluoro, methane-dichloro-fluoro, methane-difluoro, methane-difluoro-iodo, methane-disilano, methane-fluoro, methane-iodo-trifluoro, methane-nitro-trifluoro, methane-nitroso-trifluoro, methane-tetrafluoro, methane-trichlorofluoro, methane-trifluoro, methanesulfenylchloride-trifluoro, perfluoro-1-pentene, propane-1,1,1,2,2,3-hexafluoro, 2,2-difluoropropane, propane-heptafluoro-1-nitro, propane-heptafluoro-1-nitroso, propyl-1,1,1,2,3,3-hexafluoro-2,3 dichloro, 3-fluoropropylene, perfluoropropylene, 3,3,3-trifluoropropyne, 3-fluorostyrene, sulfur hexafluoride, sulfur(di)-decafluoro, trifluoroacetonitrile, trifluoromethyl peroxide, trifluoromethyl sulfide, tungsten hexafluoride, and combinations thereof. Also useful are a mixture of different types of gases, such as a perfluorocarbon gas and another type of gas such as air, or oxygen.

As mentioned above, the gas-or gas-precursor filled microspheres 100 described above are provided having incorporated therein, onto the surface thereof, and/or co-administered with, a neuromuscular blocking agent 1. For example, in one embodiment illustrated by FIG. 1, a neuromuscular blocking agent 1 is incorporated into the shell 2 of the microsphere 100.

The neurotransmitter inhibiting agent 1 is a pharmacological agent which causes neuromuscular blockade by decreasing transmission of acetylcholine. In one embodiment, this agent is botulinum toxin. Alternatively, other agents, which inhibit release or synthesis of a neurotransmitter may also be used. An examples of such an alternative agent includes hemicholinium, a synthetic compound which blocks the transport system by which choline accumulates in the terminals of the cholinergic fibers.

If botulinum toxin used, upon entering the muscular media of the vessel wall it causes a long-lasting neuromuscular blockade. Botulinum toxin type A is obtained from Allergan®. The botulinum toxin is provided in a sterile lyophilized form produced from the Hall strain of Clostridium botulinum grown in a medium containing N-Z amine and yeast extract. Botulinum toxin type A blocks neuromuscular conduction by binding to receptor sites on motor nerve terminals, entering the nerve terminals, and inhibiting the release of acetylcholine. Between 0.05 and 1000 U of botulinum toxin is delivered to the muscular media layer of a blood vessel. It has been found that a quantity in the range of between 1 and 100 U can be even more effectively used, for example, between 5 and 25 U. It is also conceived herein that more than one neuromuscular blocking agent 1 may be used in the embodiments of the present invention.

In one embodiment (not shown), the neuromuscular blocking agent is at least co-administered with the gas-or gas precursor-filled microspheres. The neuromuscular blocking agent may be applied to at least a portion of the surface of gas-or gas precursor-filled microspheres by any industrially acceptable method, including, but not limited to, spraying, waterfall coating, coating at least a portion of the surface material prior to the formation of the vesicle, or any combination and/or derivation thereof.

As mentioned above, the neuromuscular blocking agent may be incorporated into the gas-or gas precursor-filled microspheres. The process of incorporation may occur prior to, during or after the formation of the gas-filled vesicle. The neuromuscular blocking agent may be incorporated via any industrially acceptable method known to those having ordinary skill in the art. The gas-or gas precursor-filled microspheres may contain other components as well. These routes of administration for the gas-or gas precursor-filled microspheres of the present invention are those included in the above-noted patents that are incorporated by reference in their entirety.

The gas-or gas precursor-filled microspheres and neuromuscular blocking agent may be administered locally or upstream (in relation to blood flow) of the vasospasm or the site of a potential vasospasm. Via its respective mechanism of action, the neuromuscular blocking agent, or a combination of neuromuscular blocking agents may block a vasospasm prior to its occurrence and/or relieve or stops a vasospasm that is or had occurred. The composition and method disclosed herein may be utilized in any situation where a vasospasm has or may occur. Examples of these situations include, but are not limited to after a patient has a hemorrhage, a subarachnoid hemorrhage, an aneurysm, etc.

The composition of the present invention may be administered with any other known composition and/or treatment for treating vasospasms. For example, the composition of the present invention may be administered with conventional compositions and/or treatments, such as medications that increase blood pressure and/or flow to essentially force the blood through the vasospasm to the downstream tissue. Also, the composition and method of the present invention may be utilized in conjunction with an externally applied energy source such as ultrasound or the like. The externally applied energy may cause the gas-or gas precursor-filled microspheres to cavitate. This may assist, aid and/or force the neuromuscular blocking agent into the surrounding tissue to provide an enhanced localized effect.

Although the foregoing invention has been described in some detail by way of illustration and example for purposes of clarity and understanding, it will be apparent to those of ordinary skill in the art in light of the teaching of this invention that certain changes and modifications may be made thereto without departing from the spirit or scope of the claims.

What is claimed is:

1. A neuromuscular blocking preparation for blocking and/or alleviating a vasospasm, comprising:
(a) a plurality of gas-or a gas precursor-filled microspheres; and
(b) a neuromuscular blocking agent.

2. The neuromuscular blocking preparation of claim 1, wherein the neuromuscular blocking agent is incorporated within the gas-or a gas precursor-filled microspheres.

3. The neuromuscular blocking preparation of claim 1, wherein the gas-or a gas precursor-filled microspheres comprise:
(a) a shell defining a void formed within the shell; and
(b) the gas and/or a gas precursor contained within the void.

4. The neuromuscular blocking preparation of claim 1, wherein the gas-or a gas precursor-filled microspheres are fabricated of a material comprising a compound selected from a group consisting of a lipid, a liposome, a lipid coating, an emulsions, a polymer, and combinations thereof.

5. The neuromuscular blocking preparation of claim 4, wherein the lipid is a phospholipid.

6. The neuromuscular blocking preparation of claim 4, wherein the lipid is selected from a group consisting of fatty acids, lysolipids, phosphatidylcholine with saturated or unsaturated lipids, phosphatidylserine, phosphatidylglycerol, phosphatidylinositol, N-[1-(2,3-dioleoyloxy)propyl]-N,N,N-trimethylammonium chloride(DOTMA), 1,2-dioleoyloxy-3-(trimethylammonio)propane (DOTAP), 1,2-dioleoyl-3-(4′-trimethyl-ammonio)butanoyl-sn-glycerol (DOTB), sphingolipids, glycolipids, glucolipids, sulfatides, glycosphingolipids, phosphatidic acid, lipids bearing polymers, lipids bearing saccharides, lipids bearing cholesterol or derivatives thereof, tocopherol hemisuccinate, polymerized lipids, diacetyl phosphate, stearylamine, cardiolipin, phospholipids with short chain fatty acids of 6-8 carbons in length, synthetic phospholipids with asymmetric acyl chains, 6-(5-cholesten-3-β-yloxy)-1-thio-β-D-galactopyranoside, digalactosyldiglyceride, 6-(5-cholesten-3-β-yloxy)hexyl-6-amino-6-deoxy-1-thio-β-D-galacto pyranoside, 6-(5-cholesten-3-β-yloxy)hexyl-6-amino-6-deoxyl-1-thio-α-D-manno pyranoside, 12-(((7′-diethylaminocoumarin-3-yl)carbonyl)methylamino)-octadecanoic acid; N-[12-(((7′-diethylaminocoumarin-3-yl)carbonyl)methyl-amino)octadecanoyl]-2-aminopalmitic acid; cholesteryl)4′-trimethylammonio)butanoate, N-succinyldioleoylphosphatidylethanolamine, 1,2-dioleoyl-sn-glycerol, 1,2-dipalmitoyl-sn-3-succinylglycerol, 1,3-dipalmitoyl-2-succinylglycerol, 1-hexadecyl-2-palmitoylglycerophosphoethanolamine, palmitoylhomocysteine, and/or combinations thereof.

7. The neuromuscular blocking preparation of claim 6, wherein the saturated and unsaturated lipids are selected from a group consisting of dioleoylphosphatidylcholine, dimyristoylphosphatidylcholine, dipentadecanoylphosphatidylcholine, dilauroylphosphatidylcholine, dioleoylphosphatidylcholine, dipalmitoylphosphatidylcholine (DPPC), and distearoylphosphatidylcholine.

8. The neuromuscular blocking preparation of claim 6, wherein in the lipids bearing polymers, the polymer is poly(ethylene glycol).

9. The neuromuscular blocking preparation of claim 1, wherein in the gas-or a gas precursor-filled microspheres, the gas-or a gas precursor is selected from a group consisting of fluorine, perfluorocarbons, sulfur hexafluoride, hexafluoropropylene, bromochlorofluoromethane, octafluoropropane, 1,1-dichlorofluoroethane, hexafluoroethane, hexafluoro-2-butyne, perfluoropentane, octafluoro-2-butene, hexafluorobuta-1,3-diene, octafluorocyclopentene, hexafluoroacetone, tetrafluoro allene, boron trifluoride, 1,2,3-trichloro-2-fluoro-1,3-butadiene, hexafluoro-1,3-butadiene, 1-fluorobutane, decafluorobutane, perfluoro-1-butene, perfluoro-2-butene, 2-chloro-1,1,1,4,4,4-hexafluoro-butyne, perfluoro-2-butyne, octafluorocyclobutane, perfluorocyclobutene, 1,1,1-trifluorodiazoethane, hexafluorodimethyl amine, perfluorodimethylamine, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1-chloro-1,1,2,2-tetrafluoro-ethane, 2-chloro, 1,1-difluoroethane, chloropentafluoroethane, dichlorotrifluoroethane, fluoroethane, hexafluoroethane, nitropentafluoroethane, nitrosopentafluoroethane, perfluoroethylamine, 1,1-dichloro-1,2-difluoroethylene, 1,2-difluoroethylene, methane-sulfonyl chloride-trifluoro, methane-sulfonyl fluoride-trifluoro, methane-(pentafluorothio)trifluoro, methane-bromo difluoro nitroso, methane-bromo fluoro, methane-bromo chloro-fluoro, methane-bromo-trifluoro, methane-chloro difluoro nitro, methane-chloro fluoro, methane-chloro trifluoro, methane-chloro-difluoro, methane-dibromo difluoro, methane-dichloro difluoro, methane-dichloro-fluoro, methane-difluoro, methane-difluoro-iodo, methane-disilano, methane-fluoro, methane-iodo-trifluoro, methane-nitro-trifluoro, methane-nitroso-trifluoro, methane-tetrafluoro, methane-trichlorofluoro, methane-trifluoro, methanesulfenylchloride-trifluoro, perfluoro-1-pentene, propane-1,1,1,2,2,3-hexafluoro, 2,2-difluoropropane, propane-heptafluoro-1-nitro, propane-heptafluoro-1-nitroso, propyl-1,1,1,2,3,3-hexafluoro-2,3 dichloro, 3-fluoropropylene, perfluoropropylene, 3,3,3-trifluoropropyne, 3-fluorostyrene, sulfur hexafluoride, sulfur(di)-decafluoro, trifluoroacetonitrile, trifluoromethyl peroxide, trifluoromethyl sulfide, air, oxygen, and combinations thereof.

10. The neuromuscular blocking preparation of claim 9, wherein, the perfluorocarbons selected from a group consisting of perfluoromethane, perfluoroethane, perfluoropropane, perfluorobutane, perfluoropentane, perfluorohexane, perfluorocyclobutane, and combinations thereof.

11. The neuromuscular blocking preparation of claim 1, wherein the neuromuscular blocking agent selected from a group consisting of botulinum toxin, hemicholinium, and combinations thereof.

12. The neuromuscular blocking preparation of claim 1, wherein the neuromuscular blocking agent is botulinum toxin.

13. The neuromuscular blocking preparation of claim 1, wherein the gas-or a gas precursor-filled microspheres have the diameter between about 1 nanometer and about 10 micrometers.

14. The neuromuscular blocking preparation of claim 13, wherein the diameter is between about 100 nanometers and about 2 micrometers.

15. A method of treating vasospasm in a mammal, comprising administering a combination of a plurality of gas-or a gas precursor-filled microspheres and a neuromuscular blocking agent to a patient in need of treatment, thereby blocking or alleviating a vasospasm.

16. The method of claim 15, wherein the gas-or a gas precursor-filled microspheres and the neuromuscular blocking agent are administered substantially simulataneously.

17. The method of claim 15, wherein the gas-or a gas precursor-filled microspheres and the neuromuscular blocking agent are administered consecutively is a rapid succession.

18. The method of claim 15, wherein the gas-or a gas precursor-filled microspheres and the neuromuscular blocking agent are formulated into a neuromuscular blocking preparation prior to the administering.

19. The method of claim 18, wherein in the neuromuscular blocking preparation, the neuromuscular blocking agent is incorporated within the gas-or a gas precursor-filled microspheres.

20. The method of claim 15, wherein the gas-or a gas precursor-filled microspheres comprise:
(a) a shell defining a void formed within the shell; and
(b) the gas and/or a gas precursor contained within the void.

21. The method of claim 15, wherein the gas-or a gas precursor-filled microspheres are fabricated of a material comprising a compound selected from a group consisting of a lipid, a liposome, a lipid coating, an emulsions, a polymer, and combinations thereof.

22. The method of claim 21, wherein the lipid is a phospholipid.

23. The method of claim 21, wherein the lipid is selected from a group consisting of fatty acids, lysolipids, phosphatidylcholine with saturated or unsaturated lipids, phosphatidylserine, phosphatidylglycerol, phosphatidylinositol, N-[1-(2,3-dioleoyloxy)propyl]-N,N,N-trimethylammonium chloride(DOTMA), 1,2-dioleoyloxy-3-(trimethylammonio)propane (DOTAP), 1,2-dioleoyl-3-(4′-trimethyl-ammonio)butanoyl-sn-glycerol (DOTB), sphingolipids, glycolipids, glucolipids, sulfatides, glycosphingolipids, phosphatidic acid, lipids bearing polymers, lipids bearing saccharides, lipids bearing cholesterol or derivatives thereof, tocopherol hemisuccinate, polymerized lipids, diacetyl phosphate, stearylamine, cardiolipin, phospholipids with short chain fatty acids of 6-8 carbons in length, synthetic phospholipids with asymmetric acyl chains, 6-(5-cholesten-3-β-yloxy)-1-thio-β-D-galactopyranoside, digalactosyldiglyceride, 6-(5-cholesten-3-β-yloxy)hexyl-6-amino-6-deoxy-1-thio-β-D-galacto pyranoside, 6-(5-cholesten-3-β-yloxy)hexyl-6-amino-6-deoxyl-1-thio-α-D-manno pyranoside, 12-(((7′-diethylaminocoumarin-3-yl)carbonyl)methylamino)-octadecanoic acid; N-[12-(((7′-diethylaminocoumarin-3-yl)carbonyl)methyl-amino)octadecanoyl]-2-aminopalmitic acid; cholesteryl)4′-trimethylammonio)butanoate, N-succinyldioleoylphosphatidylethanolamine, 1,2-dioleoyl-sn-glycerol, 1,2-dipalmitoyl-sn-3-succinylglycerol, 1,3-dipalmitoyl-2-succinylglycerol, 1-hexadecyl-2-palmitoylglycerophosphoethanolamine, palmitoylhomocysteine, and/or combinations thereof.

24. The method of claim 23, wherein the saturated and unsaturated lipids are selected from a group consisting of dioleoylphosphatidylcholine, dimyristoylphosphatidylcholine, dipentadecanoylphosphatidylcholine, dilauroylphosphatidylcholine, dioleoylphosphatidylcholine, dipalmitoylphosphatidylcholine (DPPC), and distearoylphosphatidylcholine.

25. The method of claim 23, wherein in the lipids bearing polymers, the polymer is poly(ethylene glycol).

26. The method of claim 15, wherein in the gas-or a gas precursor-filled microspheres, the gas-or a gas precursor is selected from a group consisting of fluorine, perfluorocarbons, sulfur hexafluoride, hexafluoropropylene, bromochlorofluoromethane, octafluoropropane, 1,1-dichlorofluoroethane, hexafluoroethane, hexafluoro-2-butyne, perfluoropentane, octafluoro-2-butene, hexafluorobuta-1,3-diene, octafluorocyclopentene, hexafluoroacetone, tetrafluoro allene, boron trifluoride, 1,2,3-trichloro-2-fluoro-1,3-butadiene, hexafluoro-1,3-butadiene, 1-fluorobutane, decafluorobutane, perfluoro-1-butene, perfluoro-2-butene, 2-chloro-1,1,1,4,4,4-hexafluoro-butyne, perfluoro-2-butyne, octafluorocyclobutane, perfluorocyclobutene, 1,1,1-trifluorodiazoethane, hexafluorodimethyl amine, perfluorodimethylamine, 4-methyl-1,1,1,2-tetrafluoroethane, 1,1,1-trifluoroethane, 1,1,2,2-tetrafluoroethane, 1,1,2-trichloro-1,2,2-trifluoroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1-chloro-1,1,2,2-tetrafluoro-ethane, 2-chloro, 1,1-difluoroethane, chloropentafluoroethane, dichlorotrifluoroethane, fluoroethane, hexafluoroethane, nitropentafluoroethane, nitrosopentafluoroethane, perfluoroethylamine, 1,1-dichloro-1,2-difluoroethylene, 1,2-difluoroethylene, methane-sulfonyl chloride-trifluoro, methane-sulfonyl fluoride-trifluoro, methane-(pentafluorothio)trifluoro, methane-bromo difluoro nitroso, methane-bromo fluoro, methane-bromo chloro-fluoro, methane-bromo-trifluoro, methane-chloro difluoro nitro, methane-chloro fluoro, methane-chloro trifluoro, methane-chloro-difluoro, methane-dibromo difluoro, methane-dichloro difluoro, methane-dichloro-fluoro, methane-difluoro, methane-difluoro-iodo, methane-disilano, methane-fluoro, methane-iodo-trifluoro, methane-nitro-trifluoro, methane-nitroso-trifluoro, methane-tetrafluoro, methane-trichlorofluoro, methane-trifluoro, methanesulfenylchloride-trifluoro, perfluoro-1-pentene, propane-1,1,1,2,2,3-hexafluoro, 2,2-difluoropropane, propane-heptafluoro-1-nitro, propane-heptafluoro-1-nitroso, propyl-1,1,1,2,3,3-hexafluoro-2,3 dichloro, 3-fluoropropylene, perfluoropropylene, 3,3,3-trifluoropropyne, 3-fluorostyrene, sulfur hexafluoride, sulfur (di)-decafluoro, trifluoroacetonitrile, trifluoromethyl peroxide, trifluoromethyl sulfide, air, oxygen, and combinations thereof.

27. The method of claim 26, wherein, the perfluorocarbons selected from a group consisting of perfluoromethane, perfluoroethane, perfluoropropane, perfluorobutane, perfluoropentane, perfluorohexane, perfluorocyclobutane, and combinations thereof.

28. The method of claim 15, wherein the neuromuscular blocking agent selected from a group consisting of botulinum toxin, hemicholinium, and combinations thereof.

29. The method of claim 15, wherein the neuromuscular blocking agent is botulinum toxin.

30. The method of claim 29, wherein botulinum toxin is delivered to the muscular media layer of a blood vessel in the dosage of between about 0.05 and about 1,000 U.

31. The method of claim 30, wherein the dosage is between about 1 and about 100 U.

32. The method of claim 30, wherein the dosage is between about 5 and about 25 U.

33. The method of claim 15, wherein the gas-or a gas precursor-filled microspheres have the diameter between about 1 nanometer and about 10 micrometers.

34. The method of claim 33, wherein the diameter is between about 100 nanometers and about 2 micrometers.

35. The method of claim 15, wherein vasospasm comprises a subarachnoid induced vasospasm.
